(19) United States
(12) Patent Application Publication
(10) Pub. No.: US 2005/0196735 A1
(43) Pub. Date: Sep. 8, 2005

(54) Memory capacity tests and uses thereof

(76) Inventor: Herman Buschke, New York, NY (US)

Correspondence Address: Alan D. Miller;Amster, Rothstein & Ebenstein LLP, 90 Park Avenue, New York, NY, 10016, US

(21) Appl. No.: 11/019,928

(22) Filed: Dec. 22, 2004

Related U.S. Application Data
(60) Provisional application No. 60/535,969, filed on Jan. 12, 2004.

(51) Int. Cl. A61K 49/00; G09B 19/00

(52) U.S. Cl. 434/236; 424/9.2

(57) ABSTRACT

The present invention provides methods for assessing memory in a subject and for screening for agents directed to treating or preventing memory impairment and dementia characterized by memory impairment. The memory tests provide within-person measures of memory impairment, in addition to normative between-person measures of memory impairment, and comprise detecting memory impairment by decreased recall and discrimination of the second of two coordinated lists of items to be recalled from memory.

### CROSS-REFERENCE TO RELATED APPLICATION

This application claims the benefit of U.S. Provisional Patent Application No. 60/535,969, filed Jan. 12, 2004, the contents of which are hereby incorporated by reference into the subject application.

### STATEMENT OF GOVERNMENT INTEREST

This invention was made with government support under NIH Grant No. AG03949. As such, the United States government has certain rights in this invention.

### BACKGROUND OF THE INVENTION

It is estimated that, over the next twenty years, one in every five persons in the United States will be over the age of 65. With this new demographic profile will come an increase in a wide variety of age-related conditions, including Alzheimer's disease (“AD”) and other forms of dementia. Dementia is a syndrome of progressive decline, in multiple domains of cognitive function, that eventually leads to an inability to maintain normal social and/or occupational performance. At present, AD is the most common type of dementia, afflicting approximately 4 million Americans. One in ten persons over the age of 65, and nearly half of those over the age of 85, suffer from AD, and AD is the fourth leading cause of death in the U.S. The cost to American society is estimated to be at least $100 billion every year, making AD the third most costly disorder of aging.

Early identification is critical in progressive conditions such as AD, because earlier treatment may be more effective than later treatment in preserving cognitive function. Furthermore, early detection may allow time to explore options for treatment and care. Nevertheless, early detection is compromised by the failure of many patients to report to their treating physicians such early symptoms of AD as memory lapses and mild, but progressive, deterioration of specific cognitive functions, e.g., language (aphasia), motor skills (apraxia), and perception (agnosia). In addition, studies have documented the difficulty experienced by even well-trained health care professionals in correctly diagnosing AD and other forms of dementia (Callahan et al. 1995). Accordingly, a simple, sensitive, reliable, and easily-administered AD diagnostic test would be of great assistance in targeting individuals for early intervention.

The earliest manifestation of AD is often memory impairment—a requirement in each of the two sets of criteria for diagnosis of dementia that are commonly used: the National Institute of Neurological and Communicative Disorders and Stroke/Alzheimer's Disease and Related Disorders Association (NINCDS/ADRDA) criteria, which are specific for Alzheimer's disease, and the American Psychiatric Association's Diagnostic and Statistical Manual of Mental Disorders, 4^{th} ed. (DSM-IV) criteria, which are applicable for all forms of dementia. Therefore, any test for AD or dementia that is associated with memory impairment should be most sensitive for the early detection of memory impairment.

Conventional memory tests are not optimal for the detection of mild dementia or the early stages of Alzheimer's disease. Some of these tests are inappropriately sensitive to the patient's educational level (White and Davis, 1990). They also may fail to test for certain types of memory loss that are typical of early dementia or AD. In addition, they may fail to reflect whether compounds or therapies that are administered to treat dementia have the desired effects. Furthermore, these tests frequently suffer from a high rate of false positives (low specificity).

Despite the use of existing memory tests, the problem still to be solved is to identify people who are at a early stage of developing cognitive impairment. The generally accepted standard way to identify memory impairment is by low performance (recall, recognition, reproduction, etc.) on currently available memory tests, according to normative data. If low performance is the criterion for memory impairment, then memory impairment can only be identified when memory performance is low, necessarily making it impossible to identify less severe, earlier memory impairment. Earlier detection of cognitive impairment would permit treatment to be started at an earlier stage than is now possible, and earlier treatment may be more effective than later treatment in preserving cognitive function or in preventing further deterioration of cognitive function. Memory impairment is the earliest and most prominent sign of Alzheimer's disease. Alzheimer's disease has a very long course, where declining memory can remain within the normal range for many years. Earlier detection of memory impairment is important for earlier detection of Alzheimer's disease. Therefore, a new paradigm for assessing memory and detecting memory impairment is needed, specifically one that can detect evidence of memory impairment when declining memory is still within normal limits.

### SUMMARY OF THE INVENTION

The present invention provides memory tests that provide within-person measures of memory impairment, in addition to the standard normative between-person measures of memory impairment. The memory tests described herein are designed to detect memory impairment by decreased recall and discrimination of the second of two coordinated lists of items to be recalled from memory. These memory tests are believed to provide a more sensitive assessment of cognitive impairment than memory tests currently in general use which rely on recall of a single list.

The present invention provides a method for assessing memory in a subject, comprising the steps of: (a) presenting to the subject a first list of a plurality of items to be recalled from memory by the subject, wherein each item is from a different category; (b) having the subject recall items from the first list; (c) presenting to the subject a second list of a plurality of new items to be recalled from memory by the subject, wherein the second list comprises different items than the first list and wherein at least one item in the second list is from a category that is in the first list; (d) having the subject recall items from the second list; and (e) comparing items recalled from the second list with the items recalled from the first list, and/or adding items recalled from both lists to obtain a score and comparing the score with a score from a control population.

The present invention also provides a method for assessing memory in a subject, comprising the steps of: (a) presenting to the subject a first list of a plurality of items to be recalled from memory by the subject, wherein each item is from a different category and each item is presented using cue controlled learning where the cue comprises the category of the item; (b) having the subject recall items from the first list using cue controlled recall, where the cue comprises the category of the item; (c) presenting to the subject a second list of a plurality of new items to be recalled from memory by the subject, wherein the second list comprises different items than the first list, wherein at least one item in the second list is from a category that is in the first list, and wherein the items are presented using cue controlled learning where the cue comprises the category of the item; (d) having the subject recall items from the second list using cue controlled recall, wherein the cue comprises the category of the item; and (e) comparing items recalled from the second list with the items recalled from the first list, and/or adding items recalled from both lists to obtain a score and comparing the score with a score from a control population.

The memory tests disclosed herein can be used to assess whether a subject has a normal memory or a memory impairment.

The invention further provides methods of assessing the efficacy of an agent for treating or preventing dementia characterized by memory impairment, and the efficacy of an agent for treating memory impairment, comprising the steps of: (a) performing any of the methods disclosed herein on a subject to obtain a first score; (b) administering the agent to the subject; (c) performing the method of step (a) on the subject to obtain a second score; and (d) comparing the second score to the first score to assess the efficacy of the agent.

Additional objects and embodiments of the present invention will be apparent in view of the description which follows.

### BRIEF DESCRIPTION OF THE FIGURES

FIG. 1. Receiver Operating Characteristic (ROC) curve for subjects with clinical dementia. Compared are 1^{st} list recall, the total of 1^{st} list recall and true 2^{nd} list recall, and free recall from the Free and Cued Selective Reminding Test (FCSRT).

FIG. 2. Scatter plot of true 2^{nd} list recall versus 1^{st} list recall for normal subjects, subjects with preclinical dementia, and subjects with clinical dementia.

FIG. 3. Scatter plot of 2^{nd} list recall as a percentage of 1^{st} list recall versus free recall from the Free and Cued Selective Reminding Test (FCSRT) for normal subjects, subjects with mild cognitive impairment (MCI), and subjects with clinical dementia.

FIG. 4. Scatter plot of correct classification of list membership versus free recall from the Free and Cued Selective Reminding Test (FCSRT) for normal subjects, subjects with preclinical dementia, and subjects with clinical dementia.

FIG. 5. Scatter plot of 2^{nd} list recall as a percentage of 1^{st} list recall versus age for normal subjects, subjects with preclinical dementia, and subjects with clinical dementia.

### DETAILED DESCRIPTION OF THE INVENTION

The present invention provides a method for assessing memory in a subject, comprising the steps of: (a) presenting to the subject a first list of a plurality of items to be recalled from memory by the subject, wherein each item is from a different category; (b) having the subject recall items from the first list; (c) presenting to the subject a second list of a plurality of new items to be recalled from memory by the subject, wherein the second list comprises different items than the first list and wherein at least one item in the second list is from a category that is in the first list; (d) having the subject recall items from the second list; and (e) comparing the items recalled from the second list with the items recalled from the first list, and/or adding items recalled from both lists to obtain a score and comparing the score with a score from a control population.

As used herein, a “category” is any means of coordinating items on different lists. Items belonging to the same category share at least one common characteristic. For example, a category could correspond to a “genus,” in which case items in the category would correspond to different species within the genus. The category “animal” for example could include the items “dog” and “horse.” The category “dog” could for example include the items “poodle” and “beagle.” A category of items that are round could include “ball” and “orange.” An “item” for example can be a picture, word, phrase, sentence, or name. The items may be presented orally, in writing, in pictures, or by any other suitable means.

Items can be presented to the subject using cue controlled learning, wherein the cue comprises the category of the item. As used herein, “cue controlled learning” means that the items to be learned are associated with cues. For example, a set of items can be presented to the subject and the subject is then asked which item is associated with a specific cue. In another example, the cue and item are presented together to the subject. In the variation of the method presented in Table 4, pairs of items are sequentially presented to the subject, i.e. one pair of items followed by another pair of items.

The method can be practiced using free recall alone, cue controlled recall, or a combination of the two. “Free recall” is tested when a subject is asked to recall from memory each item presented in a list, in any order, without presentation of an associated cue or other means of assisting in recall. Items can be recalled using cue controlled recall, wherein the cue comprises the category of the item. “Cue controlled recall” means that the subject is asked to recall the items presented in the list following presentation of the associated cue.

Cue controlled learning is used to ensure the attention of the subject and the equal processing of all of the items in a list. Typically, the cue is the category that encompasses the associated item, or that defines an aspect of the associated item. For example, the cue “animal” or “stripes” might be presented in association with the item “zebra”. A cue can be anything that will elicit recall of an item and/or direct a subject's attention to an item during learning.

Categories include related associates, where one item in each list is associated with the same cue. For example, the items “diamond” and “bat” are both associated with the cue “baseball”, “November” and “turkey” are both associated with the cue “Thanksgiving”, “cow” and “tractor” are both associated with the cue “farm”, “table” and “chair” are both associated with the cue “furniture”, and “goat” and “elephant” are both associated with the cue “animal.”

The method can also be practiced using free recall followed by cued recall of items not retrieved by free recall, e.g. using the Free and Cued Selective Reminding Test (FCSRT)© (Buschke 1984). The memory component of the FCSRT comprises an initial controlled-learning step, wherein the subject first must identify items from their associated cues. Following a brief interference delay, the patient is then asked to recall as many of the presented items as possible by free recall (i.e., recall in any order, without the associated cues), followed by cued recall for items not remembered by free recall. If there are multiple trials, the subject is then selectively reminded of missed items (i.e., reminded each time an item is not recalled) before the next recall trial. The score for total recall is the total of uncued responses and cued responses, with each response (whether cued or uncued) worth one point.

The present invention also provides a method for assessing memory in a subject, comprising the steps of: (a) presenting to the subject a first list of a plurality of items to be recalled from memory by the subject, wherein each item is from a different category and each item is presented using cue controlled learning where the cue comprises the category of the item; (b) having the subject recall items from the first list using cue controlled recall, where the cue comprises the category of the item; (c) presenting to the subject a second list of a plurality of new items to be recalled from memory by the subject, wherein the second list comprises different items than the first list, wherein at least one item in the second list is from a category that is in the first list, and wherein the items are presented using cue controlled learning where the cue comprises the category of the item; (d) having the subject recall items from the second list using cue controlled recall, wherein the cue comprises the category of the item; and (e) comparing items recalled from the second list with the items recalled from the first list, and/or adding items recalled from both lists to obtain a score and comparing the score with a score from a control population.

In one embodiment of any of the methods using cue controlled recall, the subject can recall items from the first list or from the second list without using cue controlled recall, for example using free recall or free recall followed by cued recall of items not retrieved by free recall.

In one embodiment of step (e), an item recalled from the second list is only scored when the first list item in the same category is also recalled. The “control population” in step (e) can comprise subjects without memory impairment and/or subjects with memory impairment. Different populations with different degrees of memory impairment can be used, including subjects with dementia. The control population can be matched with the subject according to age, education, and/or sex.

In any of the methods using cues, the subject can be asked to recall cues using free recall.

In any of the methods described herein, the second list can comprise a different item from each category in the first list. The second list can comprise at least one new item from a category in the first list and at least one new item from a new category that is not in the first list. The presentation of second list items to the subject can comprise alternating an item from a category in the first list with an item from a category that is not in the first list. The method can comprise comparing the subject's recall of second list items in step (d) when the items are from categories in the first list versus when the items are from categories that are not in the first list.

The steps of the methods described herein can be performed in different orders, for example in the order (a), (b), (c), (d), and (e). In another embodiment of the method, step (c) is performed after step (a) and before step (b); and step (b) is performed either before step (d), after step (d), or in combination with step (d). The combination of step (b) and (d) means having the subject recall items from both the first list and from the second list. In further embodiments of the method, one or more steps can be performed more than once. For example, in one embodiment of the method, the steps are performed in order (a), (b), (c), (d), (a), (b), (c), (d), and (e). In another embodiment, the steps are performed in the order (a), (b), (c), (d); and the method further comprises after step (d) and before step (e), a step of having the subject recall items in the same category from both the first list and the second list.

In a preferred embodiment of the method, steps (a), (b), and (c) are performed in order; then a modified version of step (d) is performed wherein step (d) further comprises having the subject recall items in the same category from both the first list and from the second list, instead of having the subject recall items from only the second list; and then step (e) is performed.

In another preferred embodiment of the method, step (a) is combined with step (c), and step (b) is combined with step (d), so that the method comprises: presenting to the subject a list of a plurality of pairs of items to be recalled from memory by the subject, wherein each pair of items is from a different category and each pair is presented using cue controlled learning where the cue comprises the category of the pair of items; and having the subject recall items in each pair using cue controlled recall, where the cue comprises the category of the pair of items.

Any of the methods described herein can further comprise having the subject classify an item according to the list in which the item was presented, e.g. in the first list of items or in the second list of items.

In one embodiment of any of the methods presented herein, at least 4 categories are presented in each list. Typically, about 16 categories are presented in each list. In one embodiment, a maximum of 64 categories are presented in each list.

An item in one list can be a synonym, an antonym or an associate of an item in a second list. As used herein, “associates” are items that are clearly related to each other or to some other item. Examples of associates include “shoe” and “leather,” “table” and “chair,” and “elephant” and “rabbit.”

Items can comprise, e.g., pictures, objects, words, phrases, sentences, and/or names. Examples of objects include pencil, eraser, spoon, fork, ball, and bat.

The cues associated with presenting and/or recalling an item can comprise any one or more of, or not include any one or more of, visual, auditory, audiovisual, or tactile cues. For example, items can be presented using visual and auditory cues, but the subjects do not identify items by touch. In another example, items can be presented using only auditory cues, which for example can be used when testing a subject by telephone interview. In this example, the tester may present a first list of items by saying to the subject: “The Animal is an Elephant, The Fruit is a Banana, . . . ” When presenting the second list of items to the subject, the tester may say: “The Animal is an Cat, The Fruit is a Peach, . . . ”

Categories can be presented to the subject in random order, or in a controlled, non-random order. In one embodiment of any of the methods presented herein, items are presented to the subject using the same order of categories when each list is presented to the subject. Items can also be presented to the subject using a different order of categories when each list is presented to the subject. The same order of categories can be used during cue controlled learning and during cue controlled recall. Alternatively, a different order of categories is used during cue controlled recall than the order in which the categories were presented to the subject during cue controlled learning. For example, the order of the categories used during cue controlled recall can be reversed from the order in which the categories were presented during cue controlled learning.

The methods can comprise an additional step of “controlled rehearsal,” wherein the subject is instructed to repeat each item as it is presented. Alternately, the subject may be asked to repeat the preceding item as the current item is presented, or the subject may be instructed to repeat both the preceding item and the current item. As with cue controlled learning, controlled rehearsal ensures the attention of the subject and the equal processing of all items, and demonstrates that the required processing was performed by the subject.

In one embodiment, any of the methods presented herein comprise multiple trials of presenting items from a list to the subject and having the subject recall items from the same list, for example, multiple trials of presenting items from the first list to the subject and having the subject recall items from the first list, and/or multiple trials of presenting items from the second list to the subject and having the subject recall items from the second list.

The methods can comprise “controlled reminding,” which refers to a step wherein the subject is reminded of items that were not recalled during a trial. In one embodiment, only items that were not recalled during the immediately preceding trial are presented during a new trial. In free-recall memory tests, the subject is not reminded of the items that were not recalled until after the subject has been given the opportunity to recall as many items as possible. In the case of cued controlled recall, the subject can be reminded of the item before the next cue is presented. Controlled reminding may be either “selective reminding,” wherein the subject is reminded each time the item is not recalled, or “restricted reminding,” wherein the subject is reminded only until the item is recalled once, either with or without presentation of the item (Buschke, 1973; Spreen and Strauss, 1998).

Any of the methods can further comprises the steps of presenting to the subject one or more new list(s) of a plurality of new items to be recalled from memory, and having the subject recall items from the new list(s). The subject can be asked to recall items from all the lists that have been presented and/or to identify the list in which an item was presented. The method can further comprise comparing the items recalled from the new list(s) with the items recalled from the first list and/or the second list. In different embodiments, each item in the new list(s) is from the same category as an item in the first and second lists of items, or at least one new item in the new lists(s) is from a new category that is not in the first list or in the second list.

The recall of items from a list can be scored by counting the number of items recalled from the list or by determining the percentage of items recalled from the list. The methods can further comprise scoring items by adding the number of items recalled from the first list with the number of items recalled from the second list. In one embodiment, an item from the second list is only scored when the first list item in the same category is also recalled. As used herein, “true second list recall” refers to second list items that are recalled when their coordinated first list items for the same categories are also recalled. These scores can be compared with scores from a control population. For example, the number of items recalled from the second list when the first list item in the same category is also recalled can be added to the number of items recalled from the first list, and that combined score can be compared with the score from a control population.

The method step of comparing the items recalled from the second list with the items recalled from the first list can comprise comparing the number of second list items recalled with the number of first list items recalled or comparing the percentage of second list items recalled with the percentage of first list items recalled.

The methods can also involve measuring the number of additional items that a subject can recall from a list in response to a cue. Additional items in this context means items outside of the category with which the cue is associated.

Each item recalled by the subject can be accorded the same weight—a method known as “unit counting” or “unweighted counting”—so that a subject recalling items 1 to 5 of a ten-item list would be judged to have the same measure of memory as a subject who recalled items 6 to 10 of the same ten item list.

The recall of items can be given a weighted score when the subject recalls items in the same category from both the first list and the second list. For example, where 1 point is scored if only one of two items in a category is recalled, 3 points are scored if both items in the same category are recalled, which allows 1 additional point for recalling both items.

In one embodiment, the step of comparing the items recalled from the second list with the items recalled from the first list comprises comparing the speed with which items are recalled.

Items recalled by the subject can also be accorded a different weight depending upon some criterion. For example, items can be weighted by speed of recall or by order in a list.

Any of the methods described herein can further comprise introducing a delay after the subject recalls items from a list and then having the subject again recall items from the first list and/or second list. The method can comprise comparing items recalled after the delay with items recalled before the delay. One embodiment comprises scoring items recalled after the delay only when the same item was recalled before the delay. In one embodiment, the delay is an interference delay. As used herein, an “interference delay” is a period of time where an unrelated task (e.g., counting, spelling) is performed by the subject to prevent the subject's rehearsal of items that have been presented to the subject.

In one embodiment of methods that comprise having the subject classify an item as having been presented in the first list of items or in the second list of items, the subject is asked to classify all items that were presented to the subject. In another embodiment, the subject is only asked to classify items that are recalled by the subject. The method can comprise scoring all items that are correctly classified and/or comprise scoring when items from different lists that are in the same category are correctly classified. The subject can be asked to repeat the classification of list members to reduce chance effects. Classification can be adjusted for chance success by using methods that are generally known and used, for example, by subtracting 0.5 from the proportion of correctly classified items and multiplying the remainder by 2, where the resulting classification accuracy is scored in the range from 0.00 to 1.00 (Palfai et al. 2003).

The methods described herein can comprise having the subject recall items from one or both lists using free recall. In free recall, items can be recalled in any order. Free recall can be analyzed as the number of items recalled from each list or from both lists together, and also in terms of serial position effects (primacy vs. recency), i.e., recall as a function of serial position of an item in a list, which can be expressed as the number or % of recalls for each serial position (the classic serial position distribution, for a group of subjects), or, in a new way, as the % of each subject's recall attributed to each serial position, i.e. by calculating the percentage of each individual subject's total recall (of items recalled using free recall) for each serial position in the list (see Table 6), which provides a serial position analysis of recall for an individual subject, and in particular provides a measure of primacy for recall by an individual. Memory impairment can be shown by decreased free recall.

In the methods disclosed herein, the step of comparing items recalled from the second list with items recalled from the first list can establish that the subject has a memory impairment. One embodiment comprises comparing the number of items recalled from the second list with the number of items recalled from the first list. In one embodiment, an item recalled from the second list is only counted if the first list item in the same category is also recalled. Memory impairment can also be shown by impaired classification of items in the correct list. Those skilled in the art will also appreciate that regression analysis or logistic regression can also be used to identify an individual with memory impairment.

In one embodiment, for subjects shown to have memory impairment, the subject's score on items recalled from the second list is 60% or less of the subject's score on items recalled from the first list. In further embodiments, the subject's score on items recalled from the second list is 55% or less, 50% or less, 40% or less, or 30% or less of the subject's score on items recalled from the first list.

A subject's memory can be assessed by scoring items recalled from the first list of items or from the second list of items, and comparing the subject's score with scores from a control population. The control population can comprise subjects without memory impairment and/or subjects with memory impairment. Different populations with different degrees of memory impairment can be used, including subjects with dementia. The control population can be matched with the subject according to age, education, and/or sex. In addition, a subject's memory can be assessed by scoring items recalled from both the first and second lists of items and comparing the subject's score with scores from a control population. Typically, the number of items recalled from the first list is added to the number of items recalled from the second list. In one embodiment, an item recalled from the second list is only counted if the first list item in the same category is also recalled.

Patients in the control population can have, for example, Alzheimer's disease, or any of the other conditions associated with memory impairment which are described in this application. The subject can be assessed as having memory impairment if the subject's performance is the same as or worse than the control population having memory impairment.

The memory impairment established by any of the disclosed methods may be associated with dementia. As used herein, “dementia” refers to a chronic deterioration of cognitive skills, including intellectual function, that is severe enough to interfere with a person's ability to perform tasks and activities associated with daily living. There are many known causes of dementia, including infectious causes (e.g., bacterial endocarditis, Creutzfeldt-Jakob disease, tuberculosis and fungal meningitis, and viral encephalitis), metabolic/toxic causes (e.g., anoxia, chronic drug/alcohol/nutritional abuse, hypoglycemia, hypothyroidism, and pellagra), and structural causes (e.g., Alzheimer's disease, brain tumor, cerebellar degeneration, head trauma, Huntington's chorea, Parkinson's disease, Pick's disease, and Wilson's disease) (Beers and Berkow, 1999). The dementia can be, for example, Alzheimer's dementia, Parkinson's dementia, or Lui Body dementia.

The memory impairment may be indicative of pre-clinical Alzheimer's disease, early Alzheimer's disease, mild cognitive impairment (MCI), amnestic cognitive impairment (ACI), age-related cognitive decline, mild neurocognitive disorder, and/or delirium. The memory impairment may also be associated with a developmental defect, brain cancer, brain tumor, brain disease, traumatic brain injury, concussion, toxic exposure, infectious exposure, and/or metabolic exposure. Standard criteria for diagnoses of MCI, dementia, and major subtypes of dementia can be found in Knopman et al. (2003) and in the American Psychiatric Association's Diagnostic and Statistical Manual of Mental Disorders, 3^{rd} and 4^{th} ed.

The term “delirium”, as used herein, refers to a clinical state that is characterized by fluctuating disturbances in arousal, attention, cognition, mood, and self-awareness. Delirium generally arises acutely; it may arise without prior intellectual impairment, or it may be associated with chronic intellectual impairment (Beers and Berkow, 1999). There are many known causes of delirium, including infectious causes (e.g., acute meningitis, acute encephalitis, and infections outside of the brain), metabolic/toxic causes (e.g., anoxia, hypoglycemia, hypothyroidism, transient ischemia, and use of anticholinergic and other drugs), and structural causes (e.g., brain tumor, cerebral hemorrhage, cerebral infarction, and vascular occlusion) (Beers and Berkow, 1999).

As used herein, the term “MCI” refers to a condition of mild memory impairment that may be found in high-risk individuals with low recall. “MCI” is defined in the art to mean that a subject's recall is 1.5 standard deviations below the mean for persons of his/her age group (Petersen et al., 1999). Approximately 15% of all MCI subjects will develop dementia each year. Additionally, as used herein, the term “age-related cognitive decline” has been defined by the American Psychiatric Association's Diagnostic and Statistical Manual of Mental Disorders, 4^{th} ed. (DSM-IV) to mean that a subject's recall is 1.0 standard deviation below the norm for persons of his/her age group.

The methods disclosed herein can reveal that a subject has a memory impairment, where in contrast currently used memory tests fail to reveal a memory impairment in the same subject. In one embodiment, the subject's score on items recalled from the second list is less than (e.g., 60% or less than) the subject's score on items recalled from the first list; however, the same subject's score on the recall of the first list items indicates that the subject's memory is in a normal range. In further embodiments, the subject's score on items recalled from the second list is 55% or less, 50% or less, 40% or less, or 30% or less of the subject's score on items recalled from the first list, while the same subject's score on the recall of the first list items indicates that the subject's memory is in a normal range. Identification of memory impairment in a subject by any of the tests disclosed herein can indicate that the subject is at risk for developing clinical dementia, including Alzheimer's dementia and other clinical dementias. This is especially significant for situations where currently used memory tests fail to reveal a memory impairment in the same subject.

The invention also provides methods of assessing the efficacy of an agent for treating or preventing dementia characterized by memory impairment, comprising the steps of: (a) performing any of the methods disclosed herein on a subject to obtain a first score; (b) administering the agent to the subject; (c) performing the method of step (a) on the subject to obtain a second score; and (d) comparing the second score to the first score to assess the efficacy of the agent. The method can comprise comparing the efficacy of the agent with the efficacy of a placebo and/or with the efficacy of a second agent. The second agent, for example, may be an agent that is accepted for use for the treatment or prevention of dementia, Alzheimer's disease, and/or memory impairment. The methods may comprise administering the agent to one group of subjects and administering a placebo or second agent to a second group of subjects who are appropriately matched to the first group. The studies may also comprise a crossover design, where for example one group of subjects first receives the agent for a period of time and then receives a placebo or second agent for a period of time, while a second group of subjects first receives the placebo or second agent for a period of time and then receives the first agent for a period of time. As used herein, “treatment” of dementia includes reducing the level of dementia, preventing increases in the level of dementia, slowing down or halting the progress of dementia, attenuating the symptoms of dementia, and eliminating dementia.

Similarly, the invention also provides methods of assessing the efficacy of an agent for treating memory impairment, comprising the steps of: (a) performing any of the methods disclosed herein on a subject to obtain a first score; (b) administering the agent to the subject; (c) performing the method of step (a) on the subject to obtain a second score; and (d) comparing the second score to the first score to assess the efficacy of the agent. The memory impairment may be caused, for example, by an injury, such as a brain injury, or by a disease.

An “agent”, as used herein, can include, but not be limited to, a protein, polypeptide, peptide, nucleic acid (including DNA and/or RNA), antibody, Fab fragment, F(ab′)₂ fragment, molecule, compound, antibiotic, drug, and any combinations thereof. A Fab fragment is a univalent antigen-binding fragment of an antibody, which is produced by papain digestion. A F(ab′)₂ fragment is a divalent antigen-binding fragment of an antibody, which is produced by pepsin digestion. The antibody of the present invention may be polyclonal or monoclonal, and may be produced by techniques well known to those skilled in the art.

The methods of the present invention can also be used in selecting subjects with memory impairment for treatment trials.

The methods disclosed herein can be performed before, after or in combination with a second memory test, such as, for example, the Free and Cued Selective Reminding Test (FCSRT)© (Buschke 1984), a Free Recall test, or a Recognition test. In a Recognition test, previously tested items are mixed with new items and the subject is asked to identify those items which were previously tested and/or those items which are new items.

The memory tests disclosed herein should aid in distinguishing between subjects with true memory deficits versus individuals who seek to make exaggerated claims of their memory impairment. For example, subjects with true defects in memory impairment would be expected to have reduced recall of second list items in comparison to recall of first list items, while individuals making exaggerated or fraudulent claims of memory impairment may have similar recall of items from both lists.

The subject in the disclosed methods is preferably a mammal (e.g., an ape, a human, a lemur, a monkey, and other primates), and is most preferably a human.

Different examples of variations on the methods of the present invention are set forth below in Tables 1-6. The variation in Table 4 is designed for use, for example, over the telephone or with subjects who are visually impaired. Further variations as disclosed herein could be incorporated with the methods illustrated in Tables 1-6, for example a third list of items could be included in the method. Examples of different scoring criteria that can be used with the methods of the present invention include those summarized below in Table 5. Individual Recall Serial Position (IRSP) Curve analysis is described in Table 6.

A preferred version of the claimed method is:
a) a 1st list of items is learned and recalled by a subject; then
b) a 2nd list of items is learned but NOT recalled by the subject, then
c) items from both lists are recalled together by the subject; followed by
d) classification of the items by the subject as belonging to the first list or to the second list. This version of the method has the advantage that it is faster and more efficient than versions of the method that also include recalling the second list of items separately from recalling first list items.

In a variation of this version of the method:
a) a 1st list of items is learned and recalled by a subject; then
b) a 2nd list of items is learned but NOT recalled by the subject, then
c) items from both lists are recalled by the subject using cue controlled recall; followed by
d) free recall of items from both lists and/or free recall of cues.

Another variation that may be preferred for practical clinical use due to its simplicity is: learn 1st list of items, recall 1st list items, learn 2nd list of items, and recall 2nd list items; without recalling both list items together, and without classification of the items according to list, which may be considered optional. Another simplified variation is: learn 1st list of items, learn 2nd list of items, and recall both list items together.

Variations of the methods can be practiced using, e.g. free recall alone, or free recall followed by cued recall of items not retrieved by free recall, e.g., using the Free and Cued Selective Reminding Test (FCSRT)© (Buschke 1984), or by cued recall of all items.

The present invention is described in the following Experimental Details section, which is set forth to aid in the understanding of the invention, and should not be construed to limit in any way the scope of the invention as defined in the claims which follow thereafter.

TABLE 1
Examples of variations on Memory Capacity Tests described herein.

| | | | | Recall Both | Classify List |
|---|---|---|---|---|---|
| List 1 | | List 2 | | Lists 1 & 2 | Membership |
| Controlled | Cued | Controlled | Cued | Cued Recall | Classify |
| Learning | Recall | Learning | Recall | of Both Items | All Items |
| Controlled | Cued | Controlled | NONE | Cued Recall | Classify |
| Learning | Recall | Learning | XXXX | of BOTH Items | All Items |
| Controlled | Free * | Controlled | Free * | Free Recall | Classify |
| Learning | Recall | Learning | Recall | of ALL Items | All items |
| | | | | Followed by | |
| | | | | Cued Recall | |
| Auditory or | Free * | Auditory or | Free * | Free Recall | Classify |
| Visual | Recall | Visual | Recall | of ALL Items | All items |
| Presentation | | Presentation | | Followed by | |
| | | | | Cued Recall | |

* Free Recall of all items in List 1 or in List 2
OR: Free Recall followed by Cued Recall of ALL items in List 1 or List 2
OR: Free Recall of all items in List 1 or List 2 followed by Cued Recall of ONLY those items that were not recalled by Free Recall

Using:
category paired lists
other paired lists [associates (pencil, letter), parts of a whole (tire, windshield), etc.]

random lists

TABLE 2
Additional examples of variations on Memory Capacity Tests described herein.

| | | | | Recall Both | Free Recall of |
|---|---|---|---|---|---|
| List 1 | | List 2 | | Lists 1 & 2 | Lists 1 & 2 |
| Controlled | Cued | Controlled | Cued | Cued Recall | Free Recall of Items |
| Learning | Recall | Learning | Recall | of Both Items | and/or Cues |
| Controlled | Cued | Controlled | NONE | Cued Recall | Free Recall of Items |
| Learning | Recall | Learning | XXXX | of BOTH Items | and/or Cues |

These tests can be followed, e.g., by Individual Recall Serial Position (IRSP) Curve analysis.

TABLE 3
Additional examples of variations on Memory
Capacity Tests described herein.

| | |
|---|---|
| 1. | Learn and Recall 1st list |
| | consisting of 1 item from each of e.g. 16 (or more) |
| | different categories |
| 2. | Learn and Recall 2nd list |
| | consisting of e.g. 8 new items from 1st list categories |
| | (“2 items per cue”) |
| | alternating with e.g. 8 new items from 8 new categories |
| | (“one item per cue”) |
| 3. | In 2^{nd} List Recall |
| | compare recall of the “2 items per cue” items against the |
| | “one item per cue” items |
| | (e.g., the number of “2 items per cue” items/the number of |
| | “one item per cue” items) |

TABLE 4
Auditory variation of Memory Capacity Tests described herein.

| | |
|---|---|
| 1. | Use only ONE LIST consisting of pairs of items from each of |
| | 16 different categories, e.g. |
| | Yellow and Brown are Colors |
| | Canada and Spain are Countries |
| | Harry and Paul are Men's Names |
| | Etc . . . |
| 2. | Present the list by reading aloud to subject (Auditory) for |
| | visually impaired, or by Telephone |
| 3. | Ask subject to recall both items in each pair when presented |
| | with their Category Cue, e.g. |
| | What are the COLORS? |
| | What are the COUNTRIES? |
| | What are the MEN'S NAMES? |
| | Etc . . . |
| 4. | For Visual presentation, present in written form 2, 4, or |
| | more pairs of items from different categories, e.g.: |
| | Yellow and Brown (or Yellow/Brown) |
| | Canada and Spain |
| | Harry and Paul |
| | Present (read aloud) each Category Cue, and ask subject to |
| | identify and say aloud each pair of items in response to |
| | their Category Cue (as in #3 above) |
| 5. | Score 1 point for recall of an item by itself without |
| | recall of the other item for that cue |
| | Score 3 points for recall of both items for that cue |
| | Add scores for all cues |

TABLE 5
Examples of Different Scoring Criteria

1^{st} List Recall:

| | |
|---|---|
| a. | number of 1^{st} list items recalled |
| b. | % recalled = number of 1^{st} list items recalled/total number of |
| | list items |

2^{nd} List Recall:

| | |
|---|---|
| a. | number of 2^{nd} list items recalled |
| b. | % 2^{nd} list recall = number of 2^{nd} list items recalled/number |
| | of 1^{st} list items recalled |
| c. | true 2^{nd} list recall = number of 2^{nd} list items recalled when |
| | the 1^{st} list item from the same category also was recalled |
| d. | % true 2^{nd} list recall = number of true 2^{nd} list items recalled/ |
| | number of 1^{st} list items recalled |
| e. | joint 1^{st} & 2^{nd} recall = recall of both 1^{st} list & 2^{nd} list items |
| | from the each category |

Total 1^{st} List & 2^{nd} List Recall:

| | |
|---|---|
| a. | 1^{st} + 2^{nd} List Recall = number of 1^{st} List items recalled + |
| | number of 2^{nd} List items recalled |
| b. | 1^{st} + true 2^{nd} List recall = number of 1^{st} List items |
| | recalled + number of true 2^{nd} List items recalled |

Both 1^{st} & 2^{nd} List Recall:
recall of both 1^{st} and 2^{nd} list items together in response to each
category cue, using measures indicated above
Delayed Recall (delayed recall of both 1^{st} and 2^{nd} list items):

| | |
|---|---|
| a. | 1^{st} list delayed recall |
| b. | 2^{nd} list delayed recall |
| c. | total 1^{st} + 2^{nd} list delayed recall = 1^{st} list delayed |
| | recall + 2^{nd} list delayed recall |
| d. | % 2^{nd} list delayed recall = 2^{nd} list delayed recall/1^{st} list |
| | delayed recall |
| e. | “true 2nd” = 2^{nd} list items recalled with the 1^{st} list |
| | item from same category |
| f. | “true 2nd %” = “true” 2^{nd} list recall/1^{st} list recall in the |
| | Delayed trial |
| g. | “both 2^{nd} & 1^{st} total” = recall of both 1^{st} and 2^{nd} list |
| | items from the same category |
| h. | 1^{st} list % delayed recall = 1^{st} list delayed recall/1^{st} list |
| | recall in the “Both” condition |
| i. | 2^{nd} list % delayed recall = 2^{nd} list delayed recall/2^{nd} list |
| | recall in the “Both” condition |
| j. | total % delayed recall = total 1^{st} + 2^{nd} list delayed recall/ |
| | total 1^{st} + 2^{nd} list “Both” recall |

Delayed Retention (retention of items recalled on last trial before delay):

| | |
|---|---|
| a. | 1^{st} list retention = 1^{st} list delayed recall of items that were |
| | recalled on last trial before delay |
| b. | 2^{nd} list retention = 2^{st} list delayed recall of items that were |
| | recalled on last trial before delay |
| c. | total retention = 1^{st} list retention + 2^{nd} list retention |
| d. | % 1^{st} list retention = 1^{st} list retention/1^{st} list recall on last |
| | trial before delay |
| e. | % 2^{nd} list retention = 2^{st} list retention/2^{st} list recall on last |
| | trial before delay |
| f. | % total retention = total retention/total 1^{st} list recall + 2^{nd} |
| | list recall on last trial before delay |

Free Recall
total number recalled from first list using free recall
total number recalled from second list using free recall
total number recalled from both lists together using free recall
any of the above expressed as a % of the total recalled using free
recall, e.g. total number recalled from first list using free recall
as % of total number recalled from the first list using cued recall
Classification

| | |
|---|---|
| 1. | number of items correctly classified as belong to the list in which |
| | they were learned |
| 2. | number of items correctly classified, corrected for chance |
| 3. | number of item pairs (two items that belong to the same category) for |
| | which both items in the pair are correctly classified as belonging |
| | to the respective lists in which they were presented |
| 4. | number of item pairs that belong to the same category for which both |
| | items in the pair are correctly classified, corrected for chance |
| 5. | if classification is repeated, the number of items correctly classified |
| | in two or more repeated classification trials |
| 6. | if classification is repeated, the number of items correctly classified |
| | in two or more repeated classification trials, corrected for chance |

TABLE 6
Individual Recall Serial Position (IRSP) Curve Analysis

| | |
|---|---|
| 1. | The “serial position effect” refers to differential |
| | recall of items from a list in terms of their serial |
| | position in the list, usually graphed by a “serial |
| | position curve” that shows the number of times |
| | each item was recalled or the percentage of recall for |
| | each item, assessed by summing the recall of each |
| | item by many subjects or by summing the recall of each |
| | item over many recall trials by a single subject. |
| 2. | The classic serial position curve is characterized by |
| | recall of more items from the beginning of the |
| | list (“primacy”) and from the end of the list |
| | (“recency”) than from the middle of the list. |
| 3. | Instead of the classic serial position curve measured by |
| | the number or percentage of times each successive |
| | item in the list is recalled (based on group recall), a |
| | new kind of “Individual Recall Serial |
| | Position (IRSP) Curve” to show the relative |
| | distribution of each individual subject's recall can be |
| | constructed by calculating the percentage of each |
| | individual subject's total recall for each serial position |
| | of an item in the list: i.e., what % of each subject's |
| | recall is from each serial position (e.g., from serial |
| | position 1, from serial position 2, etc.) or what % of |
| | each subject's recall is due to recall of items from the |
| | first third, from the middle third, or from the last 3^{rd} |
| | of the list, or, alternatively, e.g., from the first, |
| | second, third, and fourth quarters of the list (to |
| | provide a serial position curve for an individual). |
| 4. | In particular, the Individual Recall Serial Position |
| | (IRSP) analysis can provide a measure of primacy for |
| | an individual, increasing detection of memory impairment. |
| 5. | Computation of Individual Recall Serial Position Curves |
| | can allow serial position analysis of recall by individual |
| | subjects, providing additional information about memory and |
| | memory impairment in individuals (as well as groups). |

### EXPERIMENTAL DETAILS

Methods

Memory tests were conducted using cue controlled learning and cue controlled recall of multiple lists of items. Cue controlled learning was carried out by presenting two or more (typically four) items (usually words or pictures) at a time, and asking the subject to identify each of the items when given its category cue (e.g., Question (from tester): “What is the animal?” Answer (from subject): “Elephant.” Question: “What is the Vegetable?” Answer: “Carrot.”). This procedure was repeated until all of the to-be-remembered items in a list were processed in this manner. Typically, 16 unrelated items, each from a different category, were used in the first list of items, and 16 different items, each from the same category as a first list item, were used in the second list of items. The category cues were used to elicit cue controlled recall of each item.

Cue controlled learning, as described above, coordinates acquisition and retrieval. This coordination is necessary in order to elicit effective cue controlled recall. Coordinated acquisition and retrieval optimize encoding specificity, and maximize cue controlled recall for more accurate detection of memory impairment. Cue controlled learning at acquisition also ensures attention and equal process of all items, shows that the subject can identify each item by its category cue, induces all subjects to process all items in the same manner on all tests, and shows that the intended processing occurred.

Following cue controlled learning of all 16 items presented to each subject, cue controlled recall of each item was tested individually using the same category cues used during cue controlled learning, to test retrieval of each of the 16 items previously presented in cue controlled learning. Such “cue controlled recall after cue controlled learning” has been shown to increase recall, and to differentially increase the recall by aged-without-dementia subjects over that by aged subjects with Alzheimer's disease, thereby increasing the effect size and improving discrimination (Buschke et al., 1997). Cue controlled learning, which induces encoding specificity, maximizes recall and should also maximize speed of recall. Cue controlled recall also assures attention and equal testing of all items, can control the order of recall, and induces all individuals to recall each item in the same way. Cue controlled learning and recall are necessary to ensure that decreased recall is due to memory impairment, and not to lack attention and/or ineffective processing strategy.

Memory was assessed using a test that included the following 6 steps:
1. Learn 1st list of 16 items, each from a different category, using cue controlled learning;
2. Recall 1st list items using cue controlled recall;
3. Learn 2nd list of 16 new items from the same 1st list categories, using cue controlled learning;
4. Recall only 2nd list items using cue controlled recall;
5. Recall both 1st and 2nd list items from the same categories using cue controlled recall; and
6. Discriminate 1st and 2nd list membership of all items.

The memory test paradigm is also indicated schematically in Table 7. Note that the test could also be performed using only steps 1-4, with the omission of step 5 and/or step 6. Memory in a normal range can be shown by recall of 1st list items (step 2), as well as by intact recall of 2nd list items in steps 4 and 5. Memory impairment can be shown by decreased recall of 2nd list items when only new 2nd list items are to be recalled (step 4) and/or by decreased recall of 2nd list items when both 1st and 2nd list items are to be recalled (step 5). Source memory impairment is shown by impaired classification of 1st and 2nd list items (step 6).

TABLE 7
Sample Memory Test Paradigm.

| 1 | 2 | 3 | 4 | 5 | 6 |
|---|---|---|---|---|---|
| | | | | 1^{st} and 2^{nd} | 1^{st} or 2^{nd} |
| 1^{st} LIST | | 2^{nd} LIST | | LIST | LIST |
| LEARN | RECALL | LEARN | RECALL | RECALL | CLASSIFY |
| 1^{st} LIST | 1^{st} LIST | 2^{nd} LIST | 2^{nd} LIST | BOTH LISTS | BY LIST |
| 1a | 1a | 1b | 1b | 1ab | 1ab |
| 2a | 2a | 2b | 2b | 2ab | 2ab |
| - | - | - | - | - | - |
| - | - | - | - | - | - |
| - | - | - | - | - | - |
| 15a | 15a | 15b | 15b | 15ab | 15ab |
| 16a | 16a | 16b | 16b | 16ab | 16ab |

“a” and “b” represent different items from each of 16 categories.

Results and Discussion

Memory was assessed in subjects with different cognitive function. Table 8 illustrates results obtained from a 92 year old, cognitively normal individual. This subject recalled all 16 items from the 1^{st} list (step 2, above) and 13 out of 16 items from the 2^{nd} list (step 4). In step 5, when both 1^{st} and 2^{nd} list items were recalled, the subject recalled 16 1^{st} list items and 15 2^{nd} list items. In step 6, the subject correctly classified all items in the correct list.

Table 9 illustrates results obtained from an 85 year old individual with clinical Alzheimer's disease. This subject recalled 10 items from the 1^{st} list (step 2) and 6 items from the 2^{nd} list (step 4). However, the subject's true 2^{nd} list recall was only 4 items, i.e. only four items were recalled from the 2^{nd} list when the item from the same category was also recalled from the first list. Thus, for this subject, the true second list recall as a percentage of 1^{st} list recall is 4/10 or 40%. In step 5, when both 1^{st} and 2^{nd} list items were recalled, the subject recalled 5 1^{st} list items and 7 2^{nd} list items, but the true 2^{nd} list recall was only 2 items. Thus, for this step, the true second list recall as a percentage of 1^{st} list recall is 2/5 or 40%. Classification performance for this subject is at chance level.

Table 10 illustrates results obtained from an 86 year old individual with ostensibly normal memory on first list recall (and FCSRT) but low 2^{nd} list recall and chance classification performance. This individual may be at increased risk for developing Alzheimer's disease.

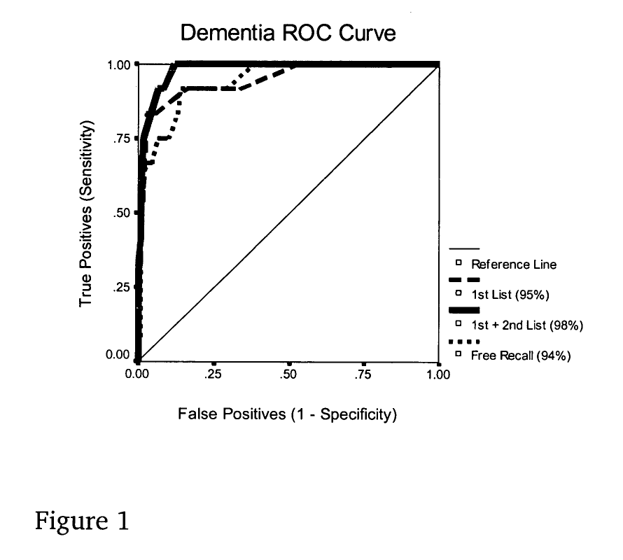

These tests are more sensitive and accurate than available memory tests that depend on free recall. FIG. 1 illustrates a Receiver Operating Characteristic (ROC) curve comparing detection of clinical dementia by the current tests and by free recall from the Free and Cued Selective Reminding Test (FCSRT) (Buschke 1984). Shown are the results from scoring 1^{st} list recall alone, the total of 1^{st} list recall plus true 2^{nd} list recall, and the results of free recall from the FCSRT. The ROC curve plots true positives (sensitivity) against false positives (1-specificity), showing the cost of increasing sensitivity in terms of increasing false alarms. A perfect sensitivity measure would hug the upper left corner. A completely inaccurate measure would follow the diagonal reference line, where every increase in sensitivity is offset by an equal increase in false alarms. The area under the ROC curve, which is the standard measure of accuracy, is 98% for total 1^{st}+true 2^{nd} list recall, 95% for 1^{st} list recall alone, and 94% for free recall from the FCSRT. All three approaches yield similar specificity of 93-94%, but the sensitivity of the total 1^{st}+true 2^{nd} list recall is 92%, whereas that of 1^{st} list recall alone is 83% and free recall is only 75%.

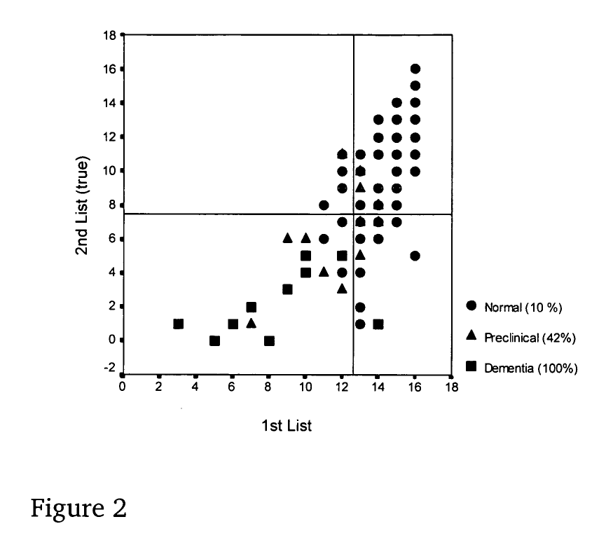

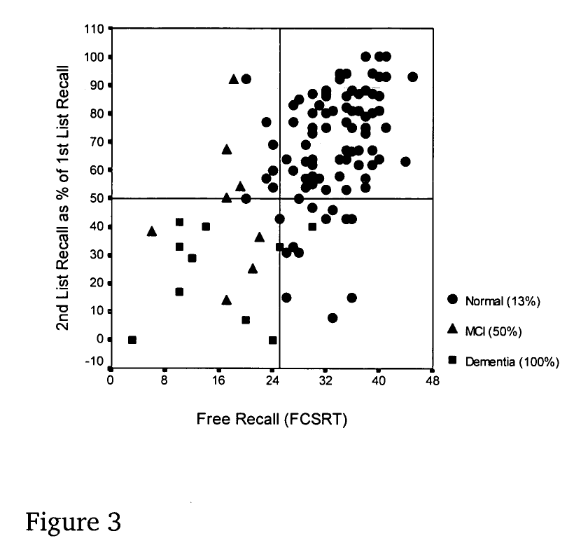

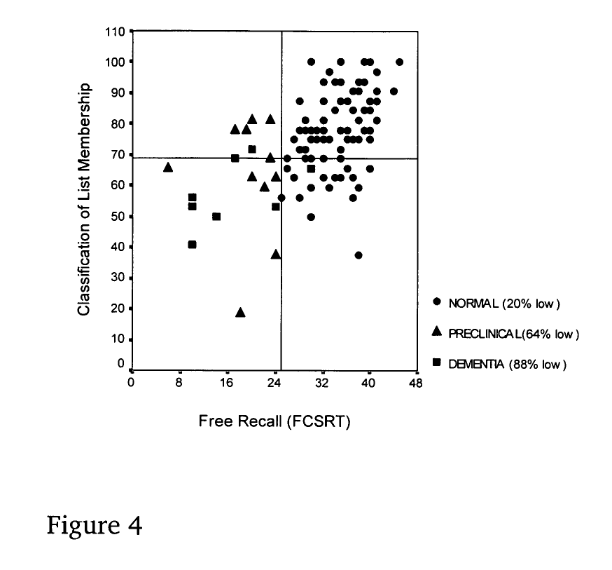

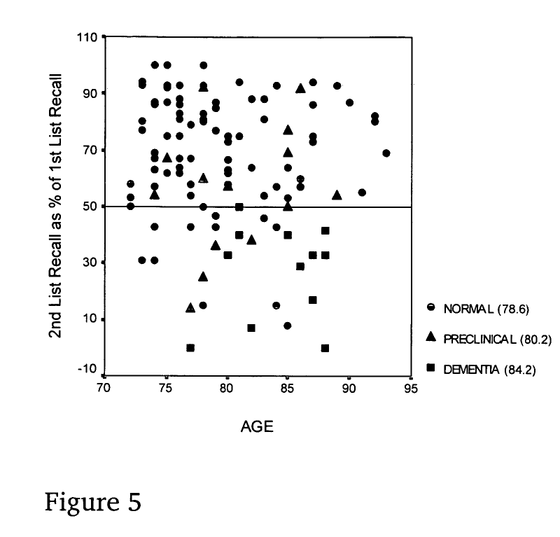

FIGS. 2-5 illustrate scatter plots of data from normal subjects, subjects with dementia and subjects with preclinical dementia or mild cognitive impairment. FIG. 2 plots true 2^{nd} list recall versus 1^{st} list recall. Recall below the reference lines indicates low recall. All subjects with dementia (DSM-III-R) scored less than 50% on true second list recall, as did 42% of subjects whose free recall on FCSRT was less than 25 and who were thus classified as preclinical subjects. 10% of normal subjects with 1^{st} list recall in the normal range also have low true second list recall. Similar results are seen in FIG. 3, which plots 2^{nd} list recall as a percentage of first list recall as a function of free recall during FCSRT. Again, all subjects with dementia scored less than 50%, as did 50% of subjects with mild cognitive impairment and 13% of normals. FIG. 4 shows scores on classification of list membership as a function of free recall during FCSRT. 88% of subjects with dementia had impaired classification as did 64% of preclinicals and 20% of normals. FIG. 5 shows that 2^{nd} list recall of 50% or less of 1st list recall occurs at all ages.

The present studies demonstrate that low 2nd list recall indicates memory impairment. 100% of persons with clinical dementia have low 2nd list recall as do 40% of persons with preclinical (MCI) 1st list recall. 10% of persons with normal range 1st list recall have low 2nd list recall; these subjects may be at risk for developing clinical dementia. Low 2nd list recall (relative to or in conjunction with 1st list recall) may facilitate earlier detection of memory impairment that is not apparent from 1st list recall alone.

TABLE 8
Memory test results from a cognitively normal individual.

| | | | | | RECALL BOTH | | CLASSIFY | |
|---|---|---|---|---|---|---|---|---|
| | 1^{st} LIST | | 2^{nd} LIST | | 1^{st} & 2^{nd} LIST | | 1^{st} & 2nd | |
| | Learn | Recall | Learn | Recall | 1st | 2nd | 1st | 2nd |
| 1 | Yellow | 1 | Brown | 1 | 1 | 1 | 1 | 1 |
| 2 | Canada | 1 | Spain | 1 | 1 | 1 | 1 | 1 |
| 3 | Dick | 1 | Paul | 1 | 1 | 1 | 1 | 1 |
| 4 | Admiral | 1 | Captain | 1 | 1 | 1 | 1 | 1 |
| 5 | Champagne | 1 | Sherry | 1 | 1 | 1 | 1 | 1 |
| 6 | Canary | 1 | Eagle | 1 | 1 | 1 | 1 | 1 |
| 7 | Linen | 1 | Silk | 1 | 1 | 1 | 1 | 1 |
| 8 | Banana | 1 | Pineapple | 1 | 1 | 1 | 1 | 1 |
| 9 | Peso | 1 | Ruble | | 1 | 1 | 1 | 1 |
| 10 | Elm | 1 | Hickory | | 1 | | 1 | 1 |
| 11 | Wall | 1 | Ceiling | 1 | 1 | 1 | 1 | 1 |
| 12 | Flea | 1 | Moth | 1 | 1 | 1 | 1 | 1 |
| 13 | Jade | 1 | Sapphire | 1 | 1 | 1 | 1 | 1 |
| 14 | Engineer | 1 | Nurse | 1 | 1 | 1 | 1 | 1 |
| 15 | Bowling | 1 | Skiing | | 1 | 1 | 1 | 1 |
| 16 | Herring | 1 | Tuna | 1 | 1 | 1 | 1 | 1 |
| Total | | 16 | | 13 | 16 | 15 | 16 | 16 |

Age 92, FCSRT score=35/48 (within normal range) (35=Free recall score, 48=Total recall score (# free recall plus cued recall of items not retrieved by free recall)).

TABLE 9
Memory test results from an individual with clinical Alzheimer's disease.

| | | | | | RECALL BOTH | | CLASSIFY | |
|---|---|---|---|---|---|---|---|---|
| | 1^{st} LIST | | 2^{nd} LIST | | 1^{st} & 2^{nd} LIST | | 1^{st} & 2nd | |
| | Learn | Recall | Learn | Recall | 1st | 2nd | 1st | 2nd |
| 1 | Yellow | 1 | Brown | | 1 | | | |
| 2 | Canada | 1 | Spain | 1 | | (1) | 1 | 1 |
| 3 | Dick | 1 | Paul | 1 | | (1) | | |
| 4 | Admiral | 1 | Captain | | | | 1 | 1 |
| 5 | Champagne | 1 | Sherry | | 1 | | | |
| 6 | Canary | 1 | Eagle | | 1 | | | (1) |
| 7 | Linen | | Silk | | | | | (1) |
| 8 | Banana | | Pineapple | | 1 | 1 | 1 | 1 |
| 9 | Peso | | Ruble | | | | (1) | |
| 10 | Elm | | Hickory | | | | 1 | 1 |
| 11 | Wall | 1 | Ceiling | | 1 | 1 | | (1) |
| 12 | Flea | | Moth | (1) | | | 1 | 1 |
| 13 | Jade | 1 | Sapphire | 1 | | (1) | 1 | 1 |
| 14 | Engineer | 1 | Nurse | | | | | (1) |
| 15 | Bowling | | Skiing | (1) | | (1) | 1 | 1 |
| 16 | Herring | 1 | Tuna | 1 | | (1) | 1 | 1 |
| Total | | 10 | | 4 | 5 | 2 | 8 | 8 |

Age 85, FCSRT score=30/43 (below normal range).

TABLE 10
Memory test results from an individual with ostensibly
normal memory on 1^{st} list recall but low 2^{nd} list
recall and chance classification performance.

| | | | | | RECALL BOTH | | CLASSIFY | |
|---|---|---|---|---|---|---|---|---|
| | 1^{st} LIST | | 2^{nd} LIST | | 1^{st} & 2^{nd} LIST | | 1^{st} & 2nd | |
| | Learn | Recall | Learn | Recall | 1st | 2nd | 1st | 2nd |
| 1 | Yellow | 1 | Brown | | | | 1 | 1 |
| 2 | Canada | 1 | Spain | 1 | 1 | 1 | 1 | 1 |
| 3 | Dick | 1 | Paul | 1 | 1 | 1 | 1 | 1 |
| 4 | Admiral | 1 | Captain | 1 | | | 1 | 1 |
| 5 | Champagne | 1 | Sherry | | 1 | | 1 | 1 |
| 6 | Canary | | Eagle | | | (1) | 1 | 1 |
| 7 | Linen | 1 | Silk | | 1 | | | (1) |
| 8 | Banana | 1 | Pineapple | 1 | 1 | 1 | 1 | 1 |
| 9 | Peso | 1 | Ruble | | 1 | | | |
| 10 | Elm | 1 | Hickory | | 1 | | 1 | 1 |
| 11 | Wall | 1 | Ceiling | 1 | | (1) | | (1) |
| 12 | Flea | 1 | Moth | | | (1) | | |
| 13 | Jade | 1 | Sapphire | | 1 | 1 | | (1) |
| 14 | Engineer | | Nurse | | | | | |
| 15 | Bowling | 1 | Skiing | 1 | 1 | 1 | | (1) |
| 16 | Herring | 1 | Tuna | | 1 | | | |
| Total | | 14 | | 6 | 10 | 5 | 8 | 8 |

Age 86, FCSRT score 35/48 (within normal range).

### REFERENCES

American Psychiatric Association, Diagnostic and Statistical Manual of Mental Disorders, 3rd ed., rev. Washington, D.C.: American Psychiatric Association, 1987.
American Psychiatric Association, Diagnostic and Statistical Manual of Mental Disorders, 4^{th} ed. Washington, D.C.: American Psychiatric Association, 1994.
Beers and Berkow (eds.), The Merck Manual of Diagnosis and Therapy, 17^{th} ed. Whitehouse Station, NJ: Merck Research Laboratories, 1999, page 1393.
Buschke, H., Selective reminding for analysis of memory and learning. Journal of Verbal Learning and Verbal Behavior, 12: 543-50, 1973.
Buschke, H. Cued recall in amnesia. J. Clinical Neuropsychology 6: 433-440, 1984.
Buschke, H. et al. Diagnosis of early dementia by the Double Memory Test: encoding specificity improves diagnostic sensitivity and specificity. Neurology, 48(4):989-97, 1997.
Callahan et al., Documentation and evaluation of cognitive impairment in elderly primary care patients. Ann. Intern. Med. 122(6):422-29, 1995.
Knopman, D. S., Boeve, B. F. and Petersen, R. C. Essentials of the proper diagnoses of mild cognitive impairment, dementia, and major subtypes of dementia. Mayo Clinic Proceedings 78: 1290-1308, 2003.
Palfai, T., Halperin, S., Hoyer, W. J. Age inequalities in recognition memory: effects of stimulus presentation time and list repetitions. Aging, Neuropsychology and Cognition. 10: 134-140, 2003.
Petersen, R., Smith, G. E., Waring, S. C., Ivnik, R. J., Tangalos, E. G., Kokmen, E. Mild cognitive impairment: clinical characterization and outcome. Archives of Neurology 56: 303-8, 1999.
Spreen O. and Strauss E. Buschke selective reminding test. In: A Compendium of Neuropsychological Tests, 2^{nd} Edition, Oxford University Press, Inc., New York, 1998, pp 282-295.
White H. and Davis P. B. Cognitive screening tests: an aid in the care of elderly outpatients. J. Gen. Intern. Med. 5:438-45, 1990.

All publications mentioned hereinabove are hereby incorporated in their entireties. While the foregoing invention has been described in some detail for purposes of clarity and understanding, it will be appreciated by one skilled in the art, from a reading of the disclosure, that various changes in form and detail can be made without departing from the true scope of the invention in the appended claims.

1. A method for assessing memory in a subject, comprising the steps of:
(a) presenting to the subject a first list of a plurality of items to be recalled from memory by the subject, wherein each item is from a different category;
(b) having the subject recall items from the first list;
(c) presenting to the subject a second list of a plurality of new items to be recalled from memory by the subject, wherein at least one item in the second list is from a category that is in the first list;
(d) having the subject recall items from the second list; and
(e) comparing the items recalled from the second list with the items recalled from the first list, and/or adding items recalled from both lists to obtain a score and comparing the score with a score from a control population.

2. The method of claim 1, wherein the second list comprises a different item from each category in the first list.

3. The method of claim 1, wherein the second list comprises at least one new item from a category in the first list and at least one new item from a new category that is not in the first list.

4. The method of claim 3, wherein presentation of second list items to the subject comprises alternating an item from a category in the first list with an item from a category that is not in the first list.

5. The method of claim 3, wherein the method further comprises comparing the subject's recall of second list items in step (d) when the items are from categories in the first list versus when the items are from categories that are not in the first list.

6. The method of claim 1, wherein the steps are performed in order (a), (b), (c), (d), and (e).

7. The method of claim 1, wherein step (c) is performed after step (a) and before step (b), and wherein step (b) is performed either before step (d), after step (d), or in combination with step (d).

8. The method of claim 1, wherein the steps are performed in order (a), (b), (c), (d), (a), (b), (c), (d), and (e).

9. The method of claim 1, wherein the steps are performed in order (a), (b), (c), (d); and wherein the method further comprises after step (d) and before step (e), a step of having the subject recall items in the same category from both the first list and the second list.

10. The method of claim 1,
wherein steps (a), (b), and (c) are performed in order;
wherein step (d) is modified to further comprise having the subject recall items in the same category from both the first list and from the second list, instead of having the subject recall items from only the second list; and
wherein step (e) is performed.

11. The method of claim 1, which further comprises having the subject classify an item as having been presented in the first list of items or in the second list of items.

12. The method of claim 1, wherein items are presented to the subject using cue controlled learning, wherein the cue comprises the category of the item.

13. The method of claim 1, wherein items are recalled by the subject using free recall.

14. The method of claim 1, wherein items are recalled by the subject using cue controlled recall, wherein the cue comprises the category of the item.

15. The method of claim 1, wherein items are recalled by the subject using free recall followed by cued recall of items not retrieved by free recall.

16. The method of claim 15, comprising a Free and Cued Selective Reminding Test (FCSRT)©.

17. A method for assessing memory in a subject, comprising the steps of:
(a) presenting to the subject a first list of a plurality of items to be recalled from memory by the subject, wherein each item is from a different category and each item is presented using cue controlled learning where the cue comprises the category of the item;
(b) having the subject recall items from the first list using cue controlled recall, where the cue comprises the category of the item;
(c) presenting to the subject a second list of a plurality of new items to be recalled from memory by the subject, wherein the second list comprises different items than the first list, wherein at least one item in the second list is from a category that is in the first list, and wherein the items are presented using cue controlled learning where the cue comprises the category of the item;
(d) having the subject recall items from the second list using cue controlled recall, wherein the cue comprises the category of the item; and
(e) comparing items recalled from the second list with the items recalled from the first list; and/or adding items recalled from both lists to obtain a score and comparing the score with a score from a control population.

18. The method of claim 17, wherein the second list comprises a different item from each category in the first list.

19. The method of claim 17, wherein the second list comprises at least one new item from a category in the first list and at least one new item from a new category that is not in the first list.

20. The method of claim 17, wherein presentation of second list items to the subject comprises alternating an item from a category in the first list with an item from a category that is not in the first list.

21. The method of claim 17, wherein the method further comprises comparing the subject's recall of second list items in step (d) when the items are from categories in the first list versus when the items are from categories that are not in the first list.

22. The method of claim 17, wherein the steps are performed in order (a), (b), (c), (d), and (e).

23. The method of claim 17, wherein step (c) is performed after step (a) and before step (b), and wherein step (b) is performed either before step (d), after step (d), or in combination with step (d).

24. The method of claim 17, wherein the steps are performed in order (a), (b), (c), (d), (a), (b), (c), (d), and (e).

25. The method of claim 17, wherein the steps are performed in order (a), (b), (c), (d); and wherein the method further comprises after step (d) and before step (e), a step of having the subject recall items in the same category from both the first list and the second list.

26. The method of claim 17,
wherein steps (a), (b), and (c) are performed in order;
wherein step (d) is modified to further comprise having the subject recall items in the same category from both the first list and from the second list, instead of having the subject recall items from only the second list; and
wherein step (e) is performed.

27. The method of claim 17, which further comprises having the subject classify an item as having been presented in the first list of items or in the second list of items.

28. The method of claim 17, wherein step (a) is combined with step (c) and step (b) is combined with step (d), wherein the method comprises:
presenting to the subject a list of a plurality of pairs of items to be recalled from memory by the subject, wherein each pair of items is from a different category and each pair is presented using cue controlled learning where the cue comprises the category of the pair of items; and
having the subject recall items in each pair using cue controlled recall, where the cue comprises the category of the pair of items.

29. The method of claim 17, which further comprises having the subject recall items from the first list and/or second list using free recall or free recall followed by cued recall of items not retrieved by free recall.

30. The method of claim 17, which further comprises having the subject recall cues using free recall.

31. The method of claim 17, wherein at least 4 categories are presented in each list.

32. The method of claim 17, wherein about 16 categories are presented in each list.

33. The method of claim 17, wherein a maximum of 64 categories are presented in each list.

34. The method of claim 17, wherein an item in one list is a synonym or an antonym of an item in the second list.

35. The method of claim 17, wherein the items comprise pictures, objects, words, phrases, sentences, and/or names.

36. The method of claim 17, wherein the cues comprise visual, auditory, audiovisual, and/or tactile cues.

37. The method of claim 17, which comprises presenting items to the subject using the same order of categories when each list is presented to the subject.

38. The method of claim 17, which comprises presenting items to the subject using a different order of categories when each list is presented to the subject.

39. The method of claim 17, which comprises using the same order of categories during cue controlled learning and during cue controlled recall.

40. The method of claim 17, which comprises using a different order of categories during cue controlled recall than the order in which categories were presented during cue controlled learning.

41. The method of claim 40, where the order of the categories during cue controlled recall is reversed from the order in which categories were presented during cue controlled learning.

42. The method of claim 17, which comprises multiple trials of presenting items from a list to the subject and having the subject recall items from the list.

43. The method of claim 42, which comprises presenting during a new trial only items that were not recalled during the immediately preceding trial.

44. The method of claim 17, which further comprises the steps of presenting to the subject one or more new list(s) of a plurality of new items to be recalled from memory, in addition to the first list of items and the second list of items, and having the subject recall items from the new list(s).

45. The method of claim 44, which further comprises having the subject identify the list in which an item was presented.

46. The method claim 44, wherein each item in the new list(s) is from the same category as an item in the first and second lists of items.

47. The method of claim 44, wherein at least one new item in the new lists(s) is from a new category that is not in the first list or in the second list.

48. The method of claim 17, wherein the step of comparing items recalled from the second list with items recalled from the first list comprises comparing the number of second list items recalled with the number of first list items recalled or comparing the percentage of second list items recalled with the percentage of first list items recalled.

49. The method of claim 17, wherein recall of an item from the second list is only scored when the first list item in the same category is also recalled.

50. The method of claim 17, wherein the step of comparing the items recalled from the second list with the items recalled from the first list comprises comparing the speed with which items are recalled.

51. The method of claim 17, which further comprises introducing a delay after the subject recalls items from a list and then having the subject again recall items from the first list and/or second list.

52. The method of claim 51, which comprises comparing items recalled after the delay with items recalled before the delay.

53. The method of claim 51, wherein the delay is an interference delay.

54. The method of claim 17, which further comprises have the subject recall items using free recall, and for items recalled by free recall, calculating the percentage of the subject's recall for each serial position in a list.

55. The method of claim 17, wherein a subject's memory is assessed by scoring items recalled from the first list of items, or from the second list of items, or from both the first and second lists of items, or by adding the number of items recalled from the second list when the first list item in the same category is also recalled with the number of items recalled from the first list, and comparing the subject's score with scores from a control population.

56. The method of claim 17, which comprises comparing performance by the subject with performance by a control population having memory impairment.

57. The method of claim 17, wherein comparing items recalled from the second list with items recalled from the first list establishes that the subject has a memory impairment.

58. The method of claim 57, which comprises comparing the number of items recalled from the second list with the number of items recalled from the first list.

59. The method of claim 57, wherein memory impairment is shown by impaired classification of items in the correct list.

60. The method of claim 57, wherein the subject's score on items recalled from the second list is 60% or less of the subject's score on items recalled from the first list.

61. The method of claim 57, wherein the subject's score on the first list of items indicates that the subject's memory is in a normal range.

62. The method of claim 57, wherein the memory impairment is associated with dementia, a developmental defect, brain cancer, brain tumor, brain disease, traumatic brain injury, concussion, toxic exposure, infectious exposure, and/or metabolic exposure, and/or wherein the memory impairment is indicative of pre-clinical Alzheimer's disease, early Alzheimer's disease, mild cognitive impairment (MCI), amnestic cognitive impairment (ACI), age-related cognitive decline, mild neurocognitive disorder, and/or delirium.

63. The method of claim 17, wherein the method is performed before, after or in combination with a second memory test.

64. The method of claim 63, wherein the second memory test is a Free and Cued Selective Reminding Test (FCSRT)©, a Free Recall test, or a Recognition Test.

65. The method of claim 17, wherein memory impairment is shown by decreased free recall.

66. The method of claim 17, wherein identification of memory impairment in the subject indicates that the subject is at risk for developing clinical dementia.

67. A method of assessing the efficacy of an agent for treating or preventing dementia characterized by memory impairment, or for treating memory impairment, comprising the steps of:
(a) performing the method of claim 17 on a subject to obtain a first score;
(b) administering an agent to the subject;
(c) performing the method of step (a) on the subject to obtain a second score; and
(d) comparing the second score to the first score to assess the efficacy of the agent.
